(19) United States
(12) Patent Application Publication
(10) Pub. No.: US 2022/0160659 A1
(43) Pub. Date: May 26, 2022

(54) PHARMACEUTICAL COMPOSITION COMPRISING a-GALACTOSYLCERAMIDE AND/OR DENDRITIC CELLS PULSED WITH a-GALACTOSYLCERAMIDE

(71) Applicant: National University Corporation Hokkaido University, Sapporo-shi (JP)

(72) Inventors: Naoki Ishimori, Sapporo-shi (JP); Toshihisa Anzai, Sapporo-shi (JP); Shintaro Kinugawa, Sapporo-shi (JP); Akimichi Saito, Sapporo-shi (JP); Yoshikuni Obata, Sapporo-shi (JP)

(73) Assignee: National University Corporation Hokkaido University, Sapporo-shi (JP)

(21) Appl. No.: 17/435,230

(22) PCT Filed: Aug. 28, 2019
(86) PCT No.: PCT/JP2019/033819

§ 371 Date: Aug. 31, 2021

(30) Foreign Application Priority Data
Mar. 4, 2019 (JP) 2019-038450

(51) Int. Cl. A61K 31/164; A61P 9/00

(52) U.S. Cl. CPC A61K 31/164; A61P 9/00

(57) ABSTRACT

The present invention pertains to a pharmaceutical composition comprising α-galactosylceramide and/or dendritic cells pulsed with α-galactosylceramide, said pharmaceutical composition being for preventing and/or treating drug-induced myocardial dysfunction. The present invention also pertains to a pharmaceutical composition comprising α-galactosylceramide and/or dendritic cells pulsed with α-galactosylceramide, said pharmaceutical composition being to be administered to a subject, who develops drug-induced myocardial dysfunction or is at a risk of developing the same, before and after administering the drug that is likely causative of the drug-induced myocardial dysfunction.

### FIELD

The present invention relates to a pharmaceutical composition for the prevention and/or treatment of drug-induced myocardial dysfunction, the pharmaceutical composition containing α-galactosylceramide (α-GalCer) and/or dendritic cells pulsed with α-GalCer.

### BACKGROUND

Cancer is a disease with the highest mortality in Japan, and it is reported that 50% of Japanese people would develop cancer. Methods for the treatment of cancer are roughly divided into surgical therapy, radiotherapy, and chemotherapy, and various advantages and disadvantages of each of the therapies have been pointed out.

Side effects of anticancer drugs are particularly regarded as problems of chemotherapy. As one of the side effects, drug-induced myocardial dysfunction can be mentioned. Drug-induced myocardial dysfunction is a disease with a basic pathological condition in which a drug causes myocardial dysfunction to exhibit cardiomyopathy. When a patient develops drug-induced myocardial dysfunction, the patient exhibits refractory heart failure and sometimes results in death. Various drugs have been reported to cause drug-induced myocardial dysfunction. In particular, an anthracycline drug, such as doxorubicin, which is a typical drug widely used as an anticancer drug, causes myocardial dysfunction from early on after the administration thereof. Even if the myocardial dysfunction is slight, with the myocardial dysfunction as an impetus, myocardial remodeling sometimes progresses in a chronic stage to cause irreversible and progressive drug-induced cardiomyopathy. For example, after cancer treatment using an anthracycline anticancer drug succeeds, the cardiac function of a patient can gradually decline, and the patient is sometimes diagnosed as drug-induced myocardial dysfunction only after the patient develops heart failure in a distant period. In contrast, immediately after a high-dose administration of an anthracycline anticancer drug, myocardial dysfunction sometimes promptly develops, in which cases even after the use of the anticancer drug is stopped, the cardiac function may not be recovered and active cancer treatment has to be given up.

The inhibition of Topoisomerase (Top) 2β activity expressed in myocardial cells, as well as the induction of, for example, DNA damage, mitochondrial dysfunction, and enhanced ROS production, which are caused by the inhibition of the Top2β activity, are thought to be important for the onset of drug-induced myocardial dysfunction caused by an anthracycline anticancer drug including doxorubicin. Recent reviews describe that drug-induced myocardial dysfunction caused by doxorubicin has a complicated mechanism involving many factors (Non Patent Literature 1). Although intervention studies have been conducted targeting Top2β and its downstream, none of them have been effective in preventing the onset or progression of drug-induced myocardial dysfunction.

Generally, the progression of heart failure is caused by a vicious circle (what is called “myocardial remodeling”) in which, with myocardial cell damage due to ischemia, pressure overload, or the like as an impetus, “the enhancement of sympathetic nervous system, renin angiotensin system, oxidative stress” caused by cardiac pump dysfunction further exacerbates the myocardial damage. The causes of heart failure are manifold, and treatment for heart failure has been mainly symptomatic treatment using, for example, an ACE inhibitor, an angiotensin receptor antagonist, or a β-blocker.

In contrast, drug-induced myocardial dysfunction is regarded as cardiomyopathy clinically similar to dilated cardiomyopathy, and also regarded as a special pathological condition involving many mechanisms inducing myocardial damage, and it is known that drug-induced myocardial dysfunction exhibits resistance against existing standard treatment for heart failure, such as treatment with an ACE inhibitor, an angiotensin receptor antagonist, a β-blocker, or the like. For example, Non Patent Literature 2 discloses that, in a pediatric cancer patient having drug-induced myocardial dysfunction caused by an anthracycline anticancer drug, the administration of ACE inhibitor enalapril reduced a left ventricular end-systolic wall stress, but failed to improve other important parameters reflecting a motor ability and the like. Non Patent Literature 3 discloses that, in an early breast cancer patient treated with an anthracycline anticancer drug, the administration of angiotensin receptor antagonist candesartan reduced a decrease in left ventricular ejection fraction, but failed to improve a left ventricular global longitudinal strain or cardiac biomarkers (troponin I, BNP), and the cardiotoxicity of anthracycline was not prevented. Non Patent Literature 3 also discloses that β-blocker metoprolol did not prevent the decrease in left ventricular ejection fraction.

From the above-described findings, drug-induced myocardial dysfunction is presumed to have a progression mechanism different from myocardial remodeling observed in typical heart failure, such as ischemic heart failure.

Furthermore, age and the presence of an underlying cardiac disease, in addition to the cumulative amount of a drug used, have been reported to contribute to the risk of developing drug-induced myocardial dysfunction, but the risk has not been able to be precisely predicted yet. With the above-described background, drug-induced myocardial dysfunction has been a hindrance to active cancer chemotherapy, and, although the prediction of onset of drug-induced myocardial dysfunction and the development of a method for the prevention or treatment thereof have been strongly socially demanded, there is no effective measure under present circumstances.

On the other hand, it is known that α-GalCer specifically activates NKT cells, and many studies on NKT cells using α-GalCer or dendritic cells pulsed with α-GalCer have been reported. The present inventors have confirmed therapeutic effects of α-GalCer (Non Patent Literature 4) and dendritic cells pulsed with α-GalCer (Patent Literature 1) on post-myocardial infarction heart failure as ischemic heart failure.

### CITATION LIST

### Patent Literature

Patent Literature 1: WO 2015/129791 pamphlet

### Non-Patent Literature

Non-Patent Literature 1: Renu et al., European Journal of Pharmacology 2018; 818: 241-253.

Non-Patent Literature 2: Silber et al., J. Clin. Oncol. 2004; 22: 820-828.

Non-Patent Literature 3: Gulati et al., European Heart Journal 2016; 37: 1671-1680.

Non-Patent Literature 4: Sobirin et al., Circ. Res. 2012; 111: 1037-1047.

### SUMMARY

### Technical Problem

An object of the present invention is to provide a means for preventing and/or treating drug-induced myocardial dysfunction.

### Solution to Problem

The present inventors found that α-GalCer and dendritic cells pulsed with α-GalCer both prevent the development of myocardial dysfunction caused by doxorubicin as an anthracycline drug or reduce the level of the myocardial dysfunction, and accomplished the following aspects of the present invention.

(1) A pharmaceutical composition for preventing and/or treating drug-induced myocardial dysfunction, the pharmaceutical composition containing α-GalCer and/or dendritic cells pulsed with α-GalCer.

(2) The pharmaceutical composition according to (1), wherein the drug-induced myocardial dysfunction is myocardial dysfunction caused by an anthracycline drug.

(3) The pharmaceutical composition according to (1), wherein the drug-induced myocardial dysfunction is myocardial dysfunction caused by doxorubicin.

(4) A pharmaceutical composition for use in a subject having developed or having the risk of developing drug-induced myocardial dysfunction, in combination with a drug having the potential of causing the drug-induced myocardial dysfunction, the pharmaceutical composition containing α-GalCer and/or dendritic cells pulsed with α-GalCer.

(5) A pharmaceutical composition for use in administering to a subject having developed or having the risk of developing drug-induced myocardial dysfunction, before and after the administration of a drug having the potential of causing the drug-induced myocardial dysfunction, the pharmaceutical composition containing α-GalCer and/or dendritic cells pulsed with α-GalCer.

(6) The pharmaceutical composition according to (4) or (5), wherein the drug having the potential of causing the drug-induced myocardial dysfunction is selected from the group consisting of an anthracycline drug, an alkylating agent, an antimetabolite, a microtubule inhibitor, and a molecular-targeted drug.

(7) The pharmaceutical composition according to (4) or (5), wherein the drug having the potential of causing the drug-induced myocardial dysfunction is an anthracycline drug.

(8) The pharmaceutical composition according to (4) or (5), wherein the drug having the potential of causing the drug-induced myocardial dysfunction is doxorubicin.

### Advantageous Effects of Invention

According to the present invention, drug-induced myocardial dysfunction that has been difficult to treat with a conventional therapeutic agent for myocardial dysfunction can be effectively prevented and/or treated.

### BRIEF DESCRIPTION OF DRAWINGS

FIG. 1 is a diagram illustrating a test schedule of α-GalCer administration to myocardial dysfunction model mice induced by doxorubicin.

FIG. 2 is a graph illustrating the left ventricular fractional shortening of the myocardial dysfunction model mice induced by doxorubicin to which α-GalCer has been administered.

FIG. 3 is a graph illustrating the ratio of myocardial tissue fibrosis of the myocardial dysfunction model mice induced by doxorubicin to which α-GalCer has been administered.

FIG. 4 is a graph illustrating the level of IL-4 gene expression in the myocardia of the myocardial dysfunction model mice induced by doxorubicin to which α-GalCer has been administered. In the figure, the gene expression level of each group is indicated as a value relative to a gene expression level in a DOX group (indicated as DOX+PBS in the figure).

FIG. 5 is a graph illustrating the levels of IFN-γ, INF-α, IL-1β, TGF-β1, IL-4, and IL-10 gene expression in the myocardia of the myocardial dysfunction model mice induced by doxorubicin to which α-GalCer has been administered. In the figure, the gene expression level of each group is indicated as a value relative to a gene expression level in a Control group.

FIG. 6 is a graph illustrating the levels of CD11c, MHC II, MCP-1, iNOS, Retnla, Arg1, and IL-1β gene expression in the myocardia of the myocardial dysfunction model mice induced by doxorubicin to which α-GalCer has been administered. In the figure, the gene expression level of each group is indicated as a value relative to a gene expression level in the Control group.

FIG. 7 is a graph illustrating the left ventricular fractional shortening of myocardial dysfunction model mice induced by doxorubicin to which dendritic cells pulsed with α-GalCer has been administered.

FIG. 8 is a Kaplan-Meier curve illustrating a survival rate of the myocardial dysfunction model mice induced by doxorubicin to which dendritic cells pulsed with α-GalCer has been administered.

### DESCRIPTION OF EMBODIMENTS

A first aspect of the present invention relates to a pharmaceutical composition for the prevention and/or treatment of drug-induced myocardial dysfunction, the pharmaceutical composition containing α-GalCer and/or dendritic cells pulsed with α-GalCer.

Alpha-GalCer used in the present invention, when presented by CD1d molecules on the cell membranes of antigen-presenting cells, such as dendritic cells, can be specifically recognized by a NKT cell receptor which is a NKT cell-specific T-cell receptor on NKT cells, and thereby can specifically activate the NKT cells. In this regard, α-GalCer itself or an analog of α-GalCer, such as a salt, ester, or derivative of α-GalCer, (for example, those described in Tashiro T., Biosci. Biotechnol. Biochem., 2012, 76(6), pp. 1055-67) can be used in the present invention. Commercial α-GalCer (for example, available from Funakoshi Co., Ltd., and REGiMMUNE Co, Ltd.) can also be used.

The dendritic cells pulsed with α-GalCer are dendritic cells obtained by a method including the step of culturing mononuclear cells in the presence of GM-CSF and IL-2 and the step of pulsing the cultured cells with α-GalCer, and a method for preparing the dendritic cells is described in, for example, WO 2015/129791, U.S. Pat. No. 10,022,401, and a literature by Ishikawa et al. (Int. J. Cancer, 2005, 117, pp. 265-273). These literatures are incorporated herein by reference in their entirety.

The culturing step is the step of culturing mononuclear cells isolated according to a conventional method, in a suitable medium containing GM-CSF and IL-2, whereby the mononuclear cells are differentiation-induced into dendritic cells. The mononuclear cells can be isolated from a variety of animal tissues, and can be typically separated, for example, from peripheral blood or apheresis cell fluid collected by a method such as density gradient centrifugation.

Considering safety upon the subsequent administration of dendritic cells, mononuclear cells are preferably collected from an animal that is the same or closely related species of the subject to which the dendritic cells are to be administered. For example, if the subject is a human, cells collected from a human as the same species are preferably used, and cells collected from a human itself to be subjected to the administration, that is, autologous mononuclear cells are more preferably used.

The medium used in the culturing step and the later-described pulsing step is a medium used usually when performing differentiation induction of mononuclear cells into dendritic cells, such as an AIM-V medium or an RPMI-1640 medium, optionally supplemented with another component, such as serum, plasma, or albumin. In the culturing step, mononuclear cells are cultured for 5 to 10 days by using the above-mentioned medium supplemented with GM-CSF adjusted to a final concentration of 500 to 1,000 U/mL, preferably approximately 800 U/mL, and IL-2 adjusted to a final concentration of 50 to 200 JRU/mL, preferably approximately 100 JRU/mL.

The pulsing step is the step of pulsing the dendritic cells prepared by the culturing step with α-GalCer. In a specific embodiment, the pulsing step is performed by culturing the dendritic cells for 8 to 48 hours in a medium containing α-GalCer at a final concentration of 50 to 200 ng/mL, preferably approximately 100 ng/mL.

The culturing step and the pulsing step may be separately performed, or the culturing step and the pulsing step may be performed at the same time by adding α-GalCer to the medium in the latter half of the culturing step.

The mononuclear cells and the dendritic cells obtained by the culturing step may be cryopreserved according to a conventional method, and thawed and used when needed in the subsequent steps. Similarly, α-GalCer-pulsed dendritic cells obtained by the pulsing step may also be cryopreserved after preparation, and thawed and used when needed.

The pharmaceutical composition according to the present invention can be administered to a subject in need of the prevention and/or treatment of drug-induced myocardial dysfunction, specifically a subject that has developed drug-induced myocardial dysfunction or has the risk of developing drug-induced myocardial dysfunction, to prevent and/or treat the drug-induced myocardial dysfunction or symptoms caused by the drug-induced myocardial dysfunction, such as cardiac hypofunction and heart failure.

The subject that has the risk of developing drug-induced myocardial dysfunction is a subject to whom a drug having the potential of causing the drug-induced myocardial dysfunction has been administered or a subject to whom the drug is scheduled to be administered. As described later, the drug having the potential of causing drug-induced myocardial dysfunction is typically an anticancer drug, and therefore, in the present invention, the subject having the risk of developing the drug-induced myocardial dysfunction is typically a cancer patient to whom the drug having the potential of causing the drug-induced myocardial dysfunction has been administered or a cancer patient to whom the drug is scheduled to be administered.

The pharmaceutical composition according to the present invention is preferably used for, particularly, a subject to whom a drug having the potential of causing drug-induced myocardial dysfunction has been administered or to whom the drug is scheduled to be administered, the subject having a high risk factor of the drug-induced myocardial dysfunction. Examples of the high risk factor of the drug-induced myocardial dysfunction caused by an anthracycline drug include: age 65 and higher; high blood pressure; a past history of cardiac disease; and recurrent cancer (See item 2.1.1.1 and Table 2 of European Heart Journal (2016) 37, 2768-2801). A subject having at least one of the above-mentioned high risk factors has the higher risk of developing drug-induced myocardial dysfunction than a subject not having any high risk factor.

Thus, the present invention also provides a pharmaceutical composition for use in a subject having developed or having the risk of developing drug-induced myocardial dysfunction, in combination with a drug having the potential of causing the drug-induced myocardial dysfunction, the pharmaceutical composition containing α-GalCer and/or dendritic cells pulsed with α-GalCer.

The prevention and/or treatment used herein covers every type of medically acceptable prophylactic and/or therapeutic intervention intended, for example, for cure, transient remission, or prevention of a disease. That is, the prevention and/or treatment of drug-induced myocardial dysfunction covers medically acceptable intervention intended for various purposes, including retardation or stop of progression of the drug-induced myocardial dysfunction, and prevention of development or prevention of recurrence of the drug-induced myocardial dysfunction.

The subject in the context of the present invention refers to any animal that may be affected with drug-induced myocardial dysfunction, and the animal is preferably a mammalian individual, for example, primates such as human and chimpanzee, rodents such as mouse, rat, guinea pig, and hamster, Artiodactyla animals such as cattle, goat, and sheep, Perissodactyla animals such as horse, and individuals of rabbit, dog, cat, and the like, and is more preferably a human individual.

Drug-induced myocardial dysfunction is myocardial dysfunction caused by the administration of a drug. Examples of a known drug having the potential of causing the drug-induced myocardial dysfunction can include: anthracycline drugs, such as doxorubicin, epirubicin, daunorubicin, idarubicin, pirarubicin, amrubicin, and mitoxantrone; alkylating agents, such as cyclophosphamide, ifosfamide, cisplatin, and mitomycin-C; antimetabolites, such as fluorouracil, capecitabine, cytarabine, and clofarabine; microtubule inhibitors, such as paclitaxel and vinca alkaloids; and molecular-targeted drugs, such as trastuzumab, bevacizumab, sunitinib, and sorafenib. The pharmaceutical composition according to the present invention is suitable for the prevention and/or treatment of myocardial dysfunction caused by an anthracycline drug or a microtubule inhibitor, preferably myocardial dysfunction caused by doxorubicin or paclitaxel, and particularly myocardial dysfunction caused by doxorubicin.

The pharmaceutical composition according to the present invention contains an effective amount of α-GalCer and/or dendritic cells pulsed with α-GalCer. The “effective amount” used herein means an amount effective for the prevention and/or treatment of drug-induced myocardial dysfunction. Such effective amount is appropriately adjusted, depending on the severity of drug-induced myocardial dysfunction, a patient, and other medical factors.

In a preferred embodiment of the pharmaceutical composition according to the present invention, an effective amount of α-GalCer is 1 to 1,000 μg, preferably 5 to 500 μg, and more preferably 10 to 100 μg/kg of body weight of an individual subjected to administration, and an effective amount of dendritic cells pulsed with α-GalCer is 10⁶ to 10⁹ cells, and preferably 10⁷ to 10⁹ cells/m² of body surface area of an individual subjected to administration. The pharmaceutical composition in such effective amounts can be administered once per day, or divided into a plurality of doses and administered, or intermittently administered.

In a preferred embodiment, the pharmaceutical composition according to the present invention is administered to a subject before and after the administration of a drug having the potential of causing drug-induced myocardial dysfunction. An illustrative regimen is as follows: the pharmaceutical composition according to the present invention is administered to a subject, typically a cancer patient, for example, a malignant lymphoma patient; several days after the administration of the pharmaceutical composition, a drug having the potential of causing drug-induced myocardial dysfunction, for example, doxorubicin or CHOP (a combination of cyclophosphamide, doxorubicin, vincristine, and prednisolone) is administered for the treatment of cancer; and furthermore, several days after the administration of the drug, the pharmaceutical composition according to the present invention is administered again.

Thus, the present invention also provides a pharmaceutical composition for use in administering to a subject having developed or having the risk of developing drug-induced myocardial dysfunction before and after the administration of a drug having the potential of causing the drug-induced myocardial dysfunction, the pharmaceutical composition containing α-GalCer and/or dendritic cells pulsed with α-GalCer

The pharmaceutical composition according to the present invention may contain a drug other than the above-mentioned active components, or pharmaceutically acceptable components, such as a buffer, an antioxidant, a preservative, a protein, a hydrophilic polymer, an amino acid, a chelating agent, a nonionic surfactant, a filler, a stabilizing agent, and a carrier. The pharmaceutically acceptable components are known to those skilled in the art, and those skilled in the art can suitably select the pharmaceutically acceptable components from, for example, those described in the revised Japanese Pharmacopoeia, 17th edition or other standards, depending on dosage forms, and used within the scope of their normal implementation ability.

The pharmaceutical composition according to the present invention can be used in the form of a parenteral preparation, such as an injection or a drip infusion. Examples of a carrier that can be used in the parenteral preparation include aqueous carriers, such as saline and an isotonic solution containing glucose, D-sorbitol, or the like.

A method for administrating the pharmaceutical composition according to the present invention is not particularly limited, but, in the case where the pharmaceutical composition is a parenteral preparation, examples of the administration method can include intravascular administration (preferably intravenous administration), intraperitoneal administration, intestinal administration, and subcutaneous administration. In one preferred embodiment, the pharmaceutical composition according to the present invention is administered to a living body by intravenous administration or intraperitoneal administration.

The present invention also provides a method for preventing and/or treating drug-induced myocardial dysfunction, the method including the step of administering an effective amount of α-GalCer and/or dendritic cells pulsed with α-GalCer to a subject that needs the prevention and/or treatment of the drug-induced myocardial dysfunction. The meanings of terms in the method for the prevention and/or treatment are the same as those described above.

The present invention will be described in more detail by the following examples, but, these examples are intended to aid the understanding of the present invention, and the technical scope of the present invention is not limited thereto.

### EXAMPLES

### Example 1

### Therapeutic Effect of α-GalCer on Myocardial Dysfunction Caused by Doxorubicin

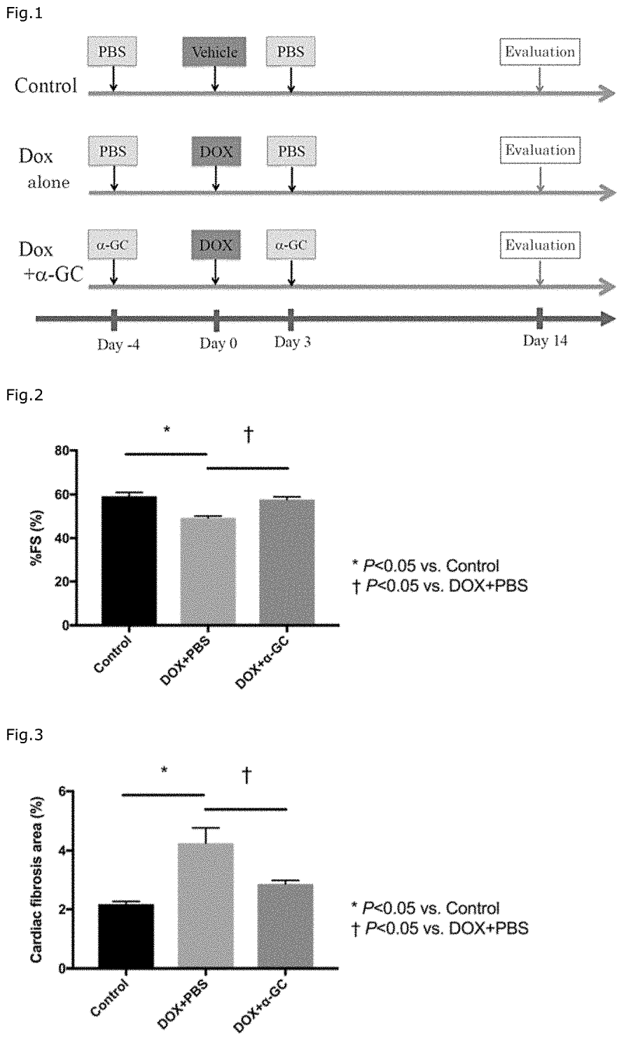

Eight-week-old male C57BL/6 mice were divided into three groups (Control group, DOX group, and DOX+α-GC group). α-GalCer (KRN7000 (Funakoshi Co., Ltd.); 0.1 μg/g weight) was intraperitoneally administered to the DOX+α-GC group, while PBS was intraperitoneally administered to the Control group and the DOX group. Four days after the administration, doxorubicin (Doxorubicin hydrochloride, D1515 (SIGMA-ALDRICH); 20 mg/kg weight) was intraperitoneally administered to the DOX group and the DOX+α-GC group, while Vehicle (PBS) was intraperitoneally administered to the Control group. Three days after the administration of DOX or PBS, α-GalCer (0.1 μg/g weight) was intraperitoneally administered to the DOX+α-GC group, while PBS was intraperitoneally administered to the Control group and the DOX group. Fourteen days after the administration of DOX or PBS, echocardiography was performed for mice of each of the groups to evaluate their cardiac functions, and subsequently, the hearts of the mice were collected to perform pathological evaluation of myocardial tissue fibrosis and evaluation of inflammatory cytokine expression by using a quantitative real-time RT-PCR method. FIG. 1 illustrates the test schedules.

The echocardiography was carried out by using an ultrasound recording device EUB-8000 (Hitachi, Ltd.) under anesthesia with pentobarbital. A left ventricular end-diastolic diameter and a left ventricular end-systolic diameter were measured from an echo image, and left ventricular fractional shortening (% FS) serving as an index of left ventricular contractility was calculated by (left ventricular end-diastolic diameter−left ventricular end-systolic diameter)/left ventricular end-diastolic diameter×100. The cardiac function (%FS) was significantly decreased in the DOX group compared to the Control group, meanwhile the DOX+α-GC group did not show the decrease in cardiac function as observed in the DOX group (see FIG. 2).

Myocardial tissue fibrosis was evaluated by fixing a frozen section sample of myocardial tissue with 10% formalin and then staining the sample with 3% Picro-sirius Red (100 ml of Van Gieson Solution P (FUJIFILM Wako Pure Chemical Corporation)+3 ml of 1% Sirius Red (MUTO PURE CHEMICALS CO., LTD.)) to detect collagen. A stained image was obtained under a microscope, and the ratio of the stained area within a region of interest to the area of the entire region of the interest was calculated. Myocardial tissue fibrosis significantly progressed in the DOX group compared with the Control group, meanwhile the progression as observed in the DOX group was significantly suppressed in the DOX+α-GC group (FIG. 3).

Furthermore, total RNA was extracted from myocardial tissues using QuickGene-810 (FUJIFILM Corporation), and subjected to a reverse transcription reaction using a cDNA reverse transcription kit (Applied Biosystems) to prepare cDNA. Using this cDNA as a template, a quantitative real-time RT-PCR was performed by the TaqMan method to evaluate the expression of inflammatory cytokine. Primer sets and probes used (all of them from Applied Biosystems) were as follows. GAPDH was used as an endogenous control.
IFN-γ: TaqMan Gene Expression Assays, Mm01168134 ml
TNF-α: TaqMan Gene Expression Assays, Mm00443258 ml
IL-1β: TaqMan Gene Expression Assays, Mm00434228 ml
TGF-β1: TaqMan Gene Expression Assays, Mm01178820 ml
IL-4: TaqMan Gene Expression Assays, Mm00445259 ml
IL-10: TaqMan Gene Expression Assays, Mm01288386 ml
GAPDH: Mouse GAPD (20X), Probe dye VIC-MGB

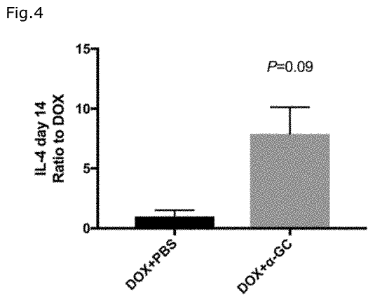

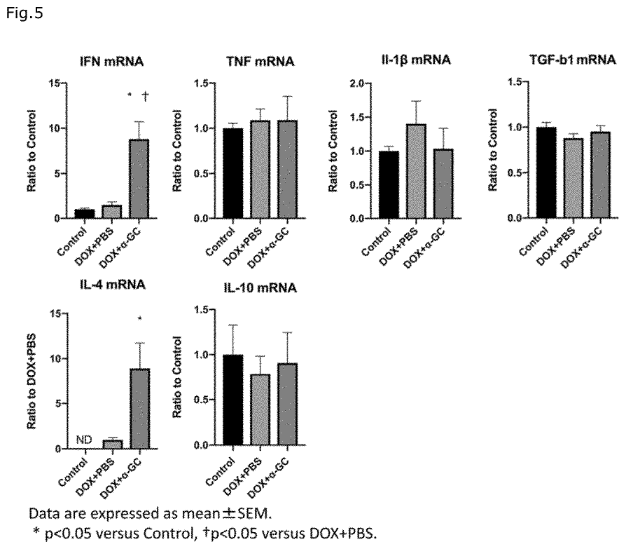

It was observed that the expression of interleukin-4 (IL-4) (FIG. 4, FIG. 5) and the expression of IFN-γ (FIG. 5) tended to be more enhanced in the DOX+α-GC group than in other groups, indicating the activation of iNKT cells in the myocardial tissues. In contrast, any significant difference in the expression of other cytokines such as IL-10 between the three groups was not observed.

Furthermore, using the above-mentioned cDNA as a template, a quantitative real-time RT-PCR was performed by the TaqMan method using the following primer sets and probes (all of them from Applied Biosystems) to evaluate the expression of CD11c and MHC II (activated macrophage markers), MCP-1, iNOS, and IL-1β (M1 macrophage markers), and Retnla and Arginase 1 (Arg1) (M2 macrophage markers). GAPDH was used as an endogenous control.
CD11c: TaqMan Gene Expression Assays, Mm00498701 ml
MHC II (H2-A1): TaqMan Gene Expression Assays, Mm00439211 ml
MCP1 (CCL2): TaqMan Gene Expression Assays, Mm00441242 ml
iNOS (NOS2): TaqMan Gene Expression Assays, Mm00440502 ml
IL-1β: TaqMan Gene Expression Assays, Mm00434228 ml
Retnla: TaqMan Gene Expression Assays, Mm00445109 ml
Arg1: TaqMan Gene Expression Assays, Mm00475988 ml
GAPDH: Mouse GAPD (20X), Probe dye VIC-MGB

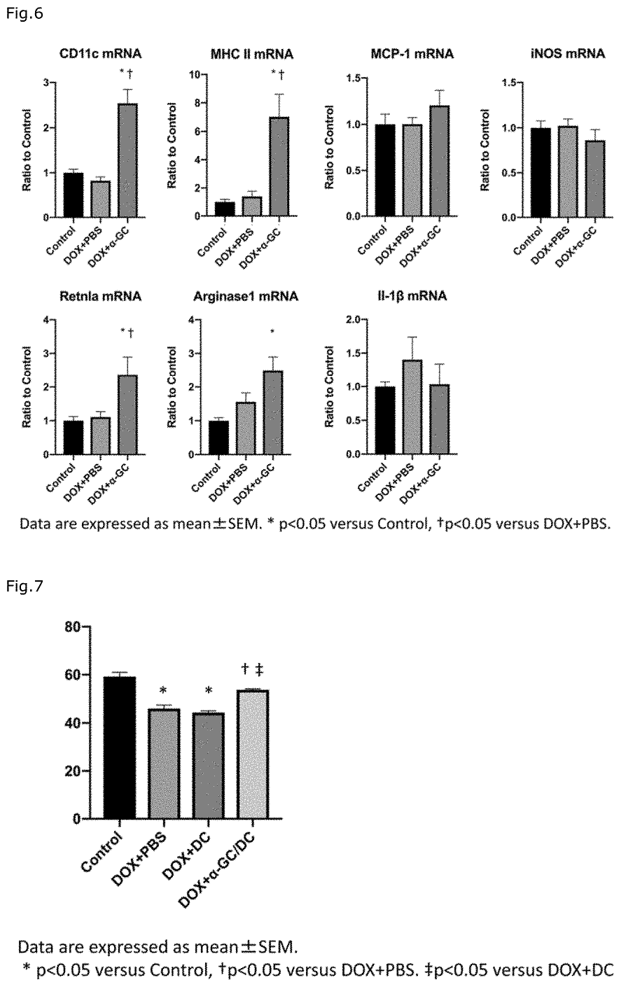

It was observed that the expression of CD11c and MHC II and the expression of Retnla and Arg1 were more enhanced in the DOX+α-GC group than in other groups. In contrast, any significant difference in the expression of MCP-1, iNOS, and IL-1β as M1 macrophage markers between the three groups was not observed (FIG. 6). From the above, it was presumed that, in the myocardial tissues, the tissue remodeling type M2 macrophages were mainly activated by the administration of α-GalCer.

### Example 2

### Therapeutic Effect of Dendritic Cells Pulsed with α-GalCer on Myocardial Dysfunction Caused by Doxorubicin

(1) Preparation of dendritic cells pulsed with α-GalCer Apheresis cell fluid was collected from healthy adult donor blood by using a continuous blood component separation device. 26.6 mL of the apheresis cell fluid was layered onto 20 mL of Ficoll-Paque PREMIUM (GE Healthcare Japan) in a 50-mL centrifuge tube, and centrifuged at 400×g for 30 minutes at 20° C. to collect a mononuclear cell layer. The mononuclear cell layer was washed with the equal volume of saline, and centrifuged at 400×g for 10 minutes at 20° C. To the resulting precipitate, an AIM-V medium supplemented with albuminate (prepared by adding 1 part by volume of 4.4% donated blood albuminate (Nippon Pharmaceutical Co., Ltd.) to 20 parts by volume of an AIM-V medium (GIBCO Invitrogen Corporation)) was added to make a suspension with volume of 45 mL, and the resulting suspension was centrifuged again. After repeating this procedure one more time, the resulting precipitate was suspended in an AIM-V medium supplemented with autologous plasma and albuminate (prepared by adding 1 part by volume of healthy adult donor plasma and 2 parts by volume of 4.4% donated blood albuminate to 40 parts by volume of an AIM-V medium), and the suspension was adjusted to have a cell concentration of 2.7×10⁸ cells/mL or less to obtain a cell suspension. 13.8 mL of the cell suspension was dispensed into 25 mL freezing bags, and 2 mL of an anti-coagulant solution, namely, ACD-A solution (Terumo Corporation) and 9.2 mL of a frost protection solution, namely, CP-1 (Kyokuto Pharmaceutical Industrial Co., Ltd.) were added to each of the bags, and then, the bags were cryopreserved at −80° C.

The cryopreserved mononuclear cells were thawed at 37° C., and washed with twice the volume of 4.4% donated blood albuminate, followed by centrifugation at 400×g for 5 minutes at 20° C. The resulting precipitate was washed again with 45 mL of saline supplemented with albuminate, followed by centrifugation to obtain mononuclear cells as a precipitate. The mononuclear cells (1×10⁸ cells) were seeded into a 225-cm² flask, and cultured at 37° C. at a CO₂ concentration of 5.0% for 7 days, using 50 mL of an AIM-V medium supplemented with autologous plasma and albuminate to which human IL-2 (Shionogi & Co., Ltd.) at final concentrations of 100 JRU/mL and human GM-CSF (North China Pharmaceutical Group Corporation-GeneTech) at a final concentration of 800 U/mL were added. On day 3 and day 6 of the culture, 50 mL of the AIM-V medium supplemented with autologous plasma and albuminate was further added, and human IL-2 and human GM-CSF were added to final concentrations of 100 JRU/mL and 800 U/mL, respectively. On day 6 of the culture, α-GalCer (Funakoshi Co., Ltd.) was added to the resultant so as to achieve a final concentration of 100 ng/mL.

After the completion of the culture, the obtained dendritic cells pulsed with α-GalCer (α-GC/DC) were collected from the flask by using a cell scraper and by pipetting, and filtered with a cell strainer, and washed with 45 mL of saline supplemented with albumin (obtained by adding 1 part by volume of 25% donated blood albumin (The Chemo-Sero-Therapeutic Research Institute) to 10 parts by volume of saline), and centrifuged at 400×g for 5 minutes at 20° C. The resulting precipitate was washed three more times in the same manner as above and suspended in 1 mL of saline supplemented with albumin, and furthermore diluted with PBS so as to achieve 3.0×10⁶/50 μL to obtain a cell suspension. A suspension of dendritic cells (DC) serving as a control was prepared in the same manner as the method described above, except that α-GalCer was not added.

(2) Evaluation of Therapeutic Effect on Myocardial Dysfunction Caused by Doxorubicin

Eight-week-old male C57BL/6 mice were divided into four groups (Control group (n=6), DOX+PBS group (n=11), DOX+DC group (n=10), and DOX+α-GC/DC group (n=10)). The DC (3×10⁶ cells per mouse) obtained in the above-described (1) was intraperitoneally administered to the DOX+DC group, the α-GC/DC (3×10⁶ cells per mouse) obtained in the above-described (1) was intraperitoneally administered to the DOX+α-GC/DC group, and PBS was intraperitoneally administered to the Control group and the DOX+PBS group. Four days after the administration, doxorubicin (20 mg/kg weight) was intraperitoneally administered to the DOX+PBS group, the DOX+DC group, and the DOX+α-GC/DC group, while vehicle (PBS) was intraperitoneally administered to the Control group. Three days after the administration of DOX or PBS, the DC (3×10⁶ cells per mouse) obtained in the above-described (1) was intraperitoneally administered to the DOX+DC group, the α-GC/DC (3×10⁶ cells per mouse) obtained in the above-described (1) was intraperitoneally administered to the DOX+α-GC/DC group, and PBS was intraperitoneally administered to the Control group and the DOX+PBS group. Fourteen days after the administration of DOX or PBS, echocardiography was performed for mice of each of the groups (Control group; n=6, DOX+PBS group; n=9, DOX+DC group; n=5, and DOX+α-GC/DC group; n=9) to evaluate their cardiac functions as in Example 1. Furthermore, during 14 days after the administration of DOX or PBS, the survival rate of the mice in each of the groups was measured.

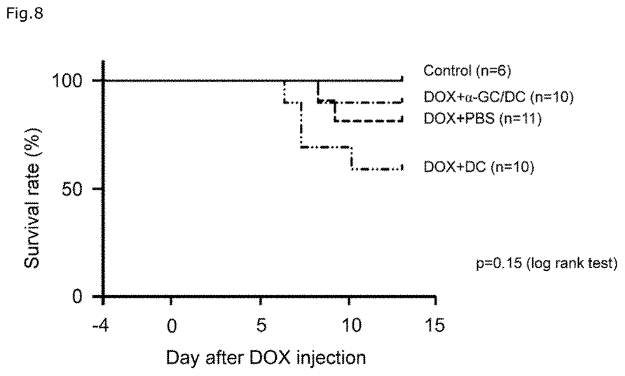

In the DOX+PBS group and the DOX+DC group, a decrease in left ventricular fractional shortening (% FS) was observed, meanwhile, in the DOX+α-GC/DC group, a decrease in left ventricular fractional shortening (% FS) was scarcely observed (FIG. 7), suggesting that dendritic cells pulsed with α-GalCer prevented a cardiac function decrease caused by the administration of doxorubicin and restored a decreased cardiac function. The survival rates in the Control group, the DOX+α-GC/DC group, the DOX+PBS group, and the DOX+DC group more greatly decreased in this order (FIG. 8).

A therapeutic effect of α-GC/DC on myocardial dysfunction caused by doxorubicin can also be confirmed in such a manner that, after the evaluation of a cardiac function, a heart is collected to perform the pathological evaluation of myocardial tissue fibrosis and the evaluation of inflammatory cytokine expression by a quantitative real-time RT-PCR method.

1-8. (canceled)

9. A method for preventing and/or treating drug-induced myocardial dysfunction, the method comprising administering an effective amount of α-galactosylceramide and/or dendritic cells pulsed with α-galactosylceramide to a subject that needs the prevention and/or treatment of the drug-induced myocardial dysfunction.

10. The method according to claim 9, wherein the drug-induced myocardial dysfunction is myocardial dysfunction caused by a drug selected from the group consisting of an anthracycline drug, an alkylating agent, an antimetabolite, a microtubule inhibitor, and a molecular-targeted drug.

11. The method according to claim 9, wherein the drug-induced myocardial dysfunction is myocardial dysfunction caused by an anthracycline drug.

12. The method according to claim 9, wherein the drug-induced myocardial dysfunction is myocardial dysfunction caused by doxorubicin.

13. The method according to claim 9, wherein the subject has developed drug-induced myocardial dysfunction, or has a risk of developing drug-induced myocardial dysfunction.

14. The method according to claim 9, wherein the subject is a cancer patient.

15. The method according to claim 9, wherein the subject is a patient to whom a drug having a potential of causing the drug-induced myocardial dysfunction has been administered, or is a patient to whom a drug having a potential of causing the drug-induced myocardial dysfunction is scheduled to be administered.

16. The method according to claim 15, wherein the drug having a potential of causing the drug-induced myocardial dysfunction is selected from the group consisting of an anthracycline drug, an alkylating agent, an antimetabolite, a microtubule inhibitor, and a molecular-targeted drug.

17. The method according to claim 15, wherein the drug having a potential of causing the drug-induced myocardial dysfunction is an anthracycline drug.

18. The method according to claim 15, wherein the drug having a potential of causing the drug-induced myocardial dysfunction is doxorubicin.

19. The method according to claim 9, further comprising administering a drug having a potential of causing the drug-induced myocardial dysfunction.

20. The method according to claim 19, wherein the effective amount of α-galactosylceramide and/or dendritic cells pulsed with α-galactosylceramide is administered before and after administering the drug having a potential of causing the drug-induced myocardial dysfunction.

21. The method according to claim 19, wherein the drug having a potential of causing the drug-induced myocardial dysfunction is selected from the group consisting of an anthracycline drug, an alkylating agent, an antimetabolite, a microtubule inhibitor, and a molecular-targeted drug.

22. The method according to claim 19, wherein the drug having a potential of causing the drug-induced myocardial dysfunction is an anthracycline drug.

23. The method according to claim 19, wherein the drug having a potential of causing the drug-induced myocardial dysfunction is doxorubicin.
